(19) United States
(12) Patent Application Publication
(10) Pub. No.: US 2021/0369595 A1
(43) Pub. Date: Dec. 2, 2021

(54) Cosmetic composition comprising an aqueous extract of rose fruit

(71) Applicant: L V M H RECHERCHE, SAINT JEAN DE BRAYE (FR)

(72) Inventors: Virginie PECHER, LA CHAPELLE SAINT MESMIN (FR); Jocelyne FRANCHI, SAINT JEAN DE LA RUELLE (FR); Marion POUJOL, ORLEANS (FR)

(73) Assignee: L V M H RECHERCHE, SAINT JEAN DE BRAYE (FR)

(21) Appl. No.: 17/048,880

(22) PCT Filed: Apr. 19, 2019
(86) PCT No.: PCT/FR2019/050941

§ 371 Date: Oct. 19, 2020

(30) Foreign Application Priority Data
Apr. 20, 2018 (FR) 1853508

(51) Int. Cl. A61K 8/9789; A61Q 19/02; A61Q 19/08; A61Q 17/00; A61Q 19/00; A61K 8/365

(52) U.S. Cl. CPC A61K 8/9789; A61Q 19/02; A61Q 19/08; A61K 2800/88; A61Q 19/007; A61K 8/365; A61K 2800/87; A61Q 17/00

(57) ABSTRACT

The present invention relates to a cosmetic composition for topical application to the skin comprising, in a physiologically acceptable medium, at least one effective amount of at least one aqueous extract of rose fruit distinct from the fruit of the dog rose (i.e. Rosa canina), in particular an aqueous extract of rose fruit of the Evanrat or Jardin de Granville® rose variety, and the use of same in particular to promote and/or improve the supply of nutrients to the skin, to strengthen the barrier function and to densify the extracellular matrix, in particular for the eye contour area.

### FIELD OF THE INVENTION

The present invention relates to a cosmetic composition comprising at least one aqueous extract of rose fruit, in particular of Jardin de Granville® rose fruit, and the use thereof in particular to promote and/or improve the supply of nutrients to the skin, to strengthen the barrier function and to densify the extracellular matrix, in particular for the eye contour area.

### PRIOR ART

It is an acknowledged fact that the quality of nutrition is reflected in the health of the skin and that certain skin disorders are associated with deficiencies, in particular of certain micronutrients (Park K. Role of micronutrients in skin health and function. Biomol. Ther. 2015; 23; 207-217).

The micronutritional balance of the skin can indeed be affected by external and/or internal factors, such as fatigue, stress, oxidative effects, UV exposure, and/or cell senescence. Observations in particular in subjects sensitive to fatigue and stress include:
a lower level of hydration (this difference increases with age);
a deficit in essential fatty acids (omega 3 and omega 6);
a deficiency of long-chain fatty acids that make up ceramides (poorer barrier function); and/or
a lower content of squalene (alteration of the protective hydrolipidic film of the cutaneous surface).

The eye contour in particular, which is the most mobile area of the face, requires twice as much energy from cells. And the Applicant identified that the fatigue of this active and fragile area led to a micronutritional imbalance contributing to the loss of radiance and the presence of bags and dark circles under the eyes.

These observations highlight imbalances, concerning micronutrients in particular.

It is known that these nutrients, or nutritional constituents, participate at the cutaneous level in particular in the establishment of an environment favorable to the reconstruction of the barrier function, to the hydration of the skin and therefore, over the long term, to skin that is resourced and less vulnerable to premature aging.

Micronutrients are essential to the development and formation of the skin (an organ undergoing constant change), to the physiological renewal of the epidermis and to the adaptation of the skin to its environment (role of protective envelope). They each have a particular role, complementary functions that cover the many facets of skin metabolism (Park K. Role of micronutrients in skin health and function. Biomol. Ther. 2015, 23: 207-217; Polefka T. Interaction of mineral salts with the skin: a literature survey. Int J. Cosmetic. Sci. 2012, 34: 416-423; Boelsma E. Nutritional skin care: health effects of micronutrients and fatty acids. Am. J. Clin. Nutr. 2001, 73: 853-864; Winkler P. Minerals and the skin in Nutrition and the skin-Lessons for anti-aging, beauty and healthy skin. 2011, Apostolos Papas Editor; Chap 7: 91-109), and chiefly:
participate in antioxidant protection (vitamins A, E, vitamin C, zinc, selenium);
are essential for the metabolism of lipids, carbohydrates or proteins (vitamins B3, B5, vitamin C for collagen synthesis), energy production (vitamin B2, magnesium);
contribute to the proper functioning of enzymes, by providing the metal ions necessary for their activity (copper, manganese, selenium, zinc, iron);
regulate epidermal differentiation (calcium);
are involved in the maintenance of membrane potential, in fluid balance, and have a role in skin hydration (potassium and sodium);
help strengthen the hydrolipidic film which helps the skin to retain its elasticity and its suppleness (Essential Fatty Acids).

The use of vitamins (A, E, C) and/or essential fatty acids is known in the formulation of cosmetic compositions intended for the skin, in particular for their protective and/or nutritional effect, but there is still a need for new compounds that make it possible to maintain and/or promote the nutritional balance of the skin and consequently stimulate hydration, the skin barrier, and/or skin regeneration, in particular for the eye contour area.

The eye contour has specific features compared with other areas of the face:
thinness of the skin to facilitate the mobility of this area (fewer basal and thorny layers than the skin of the rest of the face);
laxity (decrease in elasticity, elastic fibers, collagen) even greater in old, dry or photo-exposed skin;
dark circles (coloring of the lower eyelid); and
bags (liquid in the eyelids, loss of elasticity . . . ) increasing with age.

Furthermore, this area is rich in muscles, 22 in total, 14 of which are activated roughly every 10 seconds to ensure permanent hydration of the cornea. These perpetual movements deteriorate the fibers more rapidly, and they become less firm.
There is therefore a search for new compounds that can provide the nutrients that maintain hydration and are capable of stimulating skin metabolism, particularly in the eye contour area. The use of extracts of fruit of the dog rose (i.e. Rosa canina), also called ‘rose hips’, in moisturizing or anti-aging compositions for the face is known from the prior art.
However, the Applicant has demonstrated in the present invention the in vitro effect of an aqueous extract (obtained by means of a polar solvent) of rose fruit distinct from the rose fruit of the dog rose (i.e. Rosa canina), in particular the in vitro effect of an aqueous extract of Jardin de Granville® rose fruit, on the synthesis of ATP participating in the energy of skin cells, and on the stimulation of the expression of several genes participating in the strengthening of the barrier function and the densification of the extracellular matrix.

### SUMMARY OF THE INVENTION

A first subject matter of the invention relates to a cosmetic composition for topical application to the skin comprising, in a physiologically acceptable medium, at least one effective amount of at least one aqueous extract of rose fruit, in particular of the Evanrat or Jardin de Granville® rose variety.

Another subject matter of the invention relates to a cosmetic process intended to promote and/or improve the supply of nutrients to the skin, promote and/or improve epidermal cohesion and/or differentiation, promote and/or improve the barrier function, densify the extracellular matrix and/or reduce its degradation, improve the elasticity and/or firmness of the skin, in particular the eye contour, prevent and/or reduce dark circles and/or bags around the eyes, improve the radiance and/or homogeneity of the skin, in particular the eye contour area, promote and/or improve hydration, and/or prevent and/or reduce the formation of wrinkles and/or fine lines, comprising the application to the skin, in particular the face and/or neck and preferably the eye contour area, of a cosmetic composition as defined according to the invention.

According to a particular and preferred embodiment, the invention relates to a cosmetic process intended to prevent and/or diminish dark circles and/or bags of the eye contour area, and/or improve the radiance and/or homogeneity of the skin, in particular the eye contour, comprising the application to the eye contour of a cosmetic composition as defined according to the invention.

The invention also relates to the non-therapeutic cosmetic use of at least one effective amount of at least one aqueous extract of rose fruit according to the invention, as an agent intended to promote and/or improve the supply of nutrients to the skin, promote and/or improve epidermal cohesion and/or differentiation, promote and/or improve the barrier function, to densify the extracellular matrix and/or reduce its degradation, to improve the elasticity and/or firmness of the skin, particularly around the eyes, to prevent and/or reduce dark circles and/or bags around the eyes, to improve the radiance and/or homogeneity of the skin, particularly around the eyes, to promote and/or improve hydration, and/or to prevent and/or reduce the formation of wrinkles and/or fine lines.

According to a particular and preferred embodiment, the invention relates to the non-therapeutic cosmetic use of at least one effective amount of at least one aqueous extract of rose fruit according to the invention, as an agent intended to prevent and/or diminish dark circles and/or bags around the eyes, and/or to improve the radiance and/or homogeneity of the skin, in particular of the eye contour area.

To ‘promote and/or improve the supply of nutrients to the skin’ means to promote the supply of micronutrients present in the extract of rose fruit of the invention, such as potassium and calcium (micronutrients) and plant sugars such as fructose and glucose (macronutrients). This supply of energy (ATP synthesis) to the skin cells will help to promote the metabolism of the skin cells.

To ‘densify the extracellular matrix and/or reduce its degradation’ means to promote and/or stimulate the expression of proteins of the extracellular matrix (e.g. fibrous proteins of the collagen I and III type and elastin) which play an essential role in tissue architecture and the regulation of cell differentiation and proliferation.

To ‘promote the radiance and/or the homogeneity of the skin, in particular of the eye contour’ means in particular to improve the microcirculation and/or to decrease the vascular permeability of the skin for a better homogeneity of the complexion and a reduction in the imperfections of color of the skin (e.g. rings of the contour of the eyes).

### DETAILED DESCRIPTION OF THE INVENTION

The invention thus relates to a cosmetic composition for topical application to the skin comprising, in a physiologically acceptable medium, at least one effective amount of at least one aqueous extract of rose fruit, in particular of the Evanrat or Jardin de Granville® rose variety.

The aqueous extract of rose fruit is present in the cosmetic composition of the invention in an effective amount to obtain the desired effect.

In particular, it is present in a content ranging from 0.1% to 95%, particularly from 0.5% to 80% by weight of raw material based on the total weight of the composition. According to a first embodiment, it is present in a content ranging from 50% to 95%, in particular from 60% to 90% by weight of raw material based on the total weight of the composition. According to another particular embodiment, it is present in a content ranging from 0.1% to 20%, preferably from 0.5% to 10%, and more preferably from 1% to 5% by weight of raw material based on the total weight of the composition. Illustrative examples are given below.

Plant Material

The extracts of the invention are aqueous extracts of rose fruit. According to the invention, the terms rose extract, rosebush extract or extract in particular of the Evanrat or Jardin de Granville® rose variety are used interchangeably.
The skilled person will choose preferably selected roses whose properties are preserved by an organic environment and mode of agriculture.
In the genus Rosa, particular mention may be made of Rosa damascena, Rosa multiflora, Rosa centifolia, Rosa rugosa, Rosa chinensis, Rosa moschata, Rosa alba, Rosa alpina, Rosa cinnamonea, Rosa gallica, Rosa repens, Rosa rubrifolia, Rosa rubiginosa, Rosa sempervirens, Rosa spinosissima, Rosa stylosa, Rosa tomentosa, or Rosa villosa, excluding the species Rosa canina (dog rose).
According to a particular and preferred embodiment, an aqueous extract of rose fruit of the Evanrat or ‘Jardin de Granville®’ rose variety will be used.

The ‘Jardin de Granville®’ rosebush or rose is a hybrid variety offered exclusively by “Roses anciennes André Eve S.A.S.” and protected by plant breeder's rights under No. 20110345 with the name Rosa L. for the species and the name EVANRAT for the variety. This rose bush belongs to the group of modern hybrids, which, from May to October, is permanently covered with roses, showing excellent flowering character.
Thus, according to a particular and preferred embodiment of the invention, the cosmetic composition according to the invention includes an aqueous extract of rose fruit of the Evanrat or Jardin de Granville® rose variety.

The fruit of the rose according to the invention are distinct from the fruit of the dog rose (i.e. Rosa canina), which are called rose hips.

The rose fruit according to the invention are obtained by letting the flower wither on the rosebush. Three types of rose fruit can be distinguished according to their maturity:
the ‘green’ fruit harvested in October and resulting from a second flowering in September;
the ‘intermediate’ fruit harvested in October and resulting from a second flowering in August; and
the ‘ripe’ fruit (red-orange color) harvested in October and resulting from a first flowering in June.

According to the invention, rose fruit will be used, chosen respectively from ‘green’ fruit, ‘intermediate’ fruit, ‘ripe’ fruit, and mixtures thereof.

According to a particular embodiment of the invention, ‘green’ fruit will be used.

According to another particular embodiment of the invention, ‘intermediate’ fruit will be used.

According to yet another particular embodiment of the invention, ‘ripe’ fruit will be used.

In the context of the invention and according to another particular embodiment, ‘intermediate’ and ‘ripe’ fruit will be used.

Green, intermediate and/or ripe fruits are referred to in the rest of the description as rose ‘fruit’. These rose fruits are usually harvested in October and immediately stored at −20° C.

Whole fruit including the cap, hairs and pulp can be used, or the pulp alone.

These rose fruits include monosaccharide sugars (fructose, glucose, sucrose), organic acids (citric acid, malic acid), polyphenols (catechin), vitamin C, amino acids (mainly aspartic acid and glutamic acid), minerals (ash, potassium, calcium), and carotenoids.

In particular, calcium improves epidermal differentiation and strengthens the structure of the skin, while potassium amplifies skin hydration and boosts energy assimilation. Plant sugars (fructose, sucrose, saccharose) are intended to fill the skin cells with energy.

The rose fruit of the invention can be harvested and directly frozen; these will be referred to as ‘wet’ or ‘fresh’ frozen fruit. According to another embodiment, the rose fruit of the invention can be harvested and dried (freeze-dried) before being frozen; these will be referred to as frozen ‘freeze-dried’ fruit.

According to a particular and preferred embodiment, ‘wet’ or ‘fresh’ frozen fruit will be used for extraction.

The aqueous extract of rose fruit according to the invention is an extract concentrated in polar natural compounds. The aqueous extract of rose fruit according to the invention is distinct from rose water. This is a extract of rose fruit involving the extraction of natural rose fruit compounds in the presence of aqueous (polar) solvents with or without the addition of a pH modifier, and the use, in the formulation, in the form of an aqueous solution or concentrate, in which the solvent of the concentrate may be the extraction solvent and/or an additional solvent.

According to a particular embodiment, the aqueous extract of rose fruit according to the invention further comprises in a small proportion an extract of rose flowers representing from 0.01 to 1%, preferably from 0.01 to 0.1% of the total aqueous extract of rose according to the invention. In this embodiment, the original plant material (10% of the total load of plant material) comprises rose fruits and rose flowers in a proportion of 0.01% flowers per 9.99% fruit to 1% flowers per 9% fruit.

The inventors have indeed demonstrated that the aqueous extracts of rose fruit according to the invention included micronutrients and natural compounds of interest for the skin.

By way of natural polar compounds of interest present in the aqueous extract of rose fruit according to the invention, particular mention may be made of:
sugars (glucose, fructose, possibly saccharose) and vitamin C, at the core of the skin's energy processes,
organic acids (citric acid, malic acid), polyphenols (catechin), carriers of anti-radical activities,
minerals, combined with skin protection and repair systems, and
amino acids, source of vitality and protein constituents for the cell.

The aqueous extract of rose fruit according to the invention may be obtained by various processes known to the skilled person and in particular those described below.

According to a particular and preferred embodiment, fruit of the Evanrat variety, preferably the Jardin de Granville® rose, preferably fresh fruit directly frozen at −20° C., will be used as plant material in the extraction processes adapted to the invention described below.

Aqueous Extract of Rose Fruit

The terms aqueous extract or polar extract or hydrophilic extract of rose are used interchangeably in the description. Aqueous extract of rose fruit is obtained using a cosmetically acceptable polar solvent.

The term ‘aqueous extract of rose’ according to the invention means in particular that the polar (hydrophilic) compounds of the rose fruit have been solubilized and/or extracted in a polar solvent.

Advantageously, the extract of the invention is obtained by ‘Eco extraction’ based on the discovery and design of extraction processes to reduce energy consumption, but also the use of alternative solvents and renewable plant resources, while ensuring a safe and quality product/extract. Eco-extraction is based on 6 main principles (Farid Chemat, Dunod, 2010, Eco-Extraction du végétal):
Promote innovation through varietal selection and the use of renewable plant resources,
Prefer alternative solvents known as green solvents and mainly those from agro-resources,
Reduce energy consumption through the assistance of innovative technologies and promote energy recovery,
Encourage the creation of co-products instead of waste to integrate the organic or agro-refinery route,
Reduce unit operations through technological innovation and promote safe, robust and controlled processes, and
Prefer a product that is undenatured, biodegradable, contaminant-free and above all, has “eco-Extract” values. It is therefore advantageous to use water as a polar green solvent.

Prior to the extraction step itself, the fruits may have been dried and/or ground. According to another embodiment, the fruits are used wet and then ground.

According to a particular embodiment, the fruits are ground before extraction, for example by means of a mortar, a cryo-grinder, a mixer, a traditional mill or a centrifuge according to the methods known to the skilled person.

The extract can be prepared by various extraction processes known to the skilled person, involving the steps of grinding the plant material, dispersion of the ground material in a polar solvent, separation of the soluble and insoluble phases by filtration, concentration and possible re-solution.

According to the present invention, the expression “polar solvent” means that the solvent has a polarity index value or Hildebrand total solubility parameter greater than or equal to 10. The polarity index is a quantity calculated on the basis of thermodynamic quantities (of solubility and change of state) which highlights the more or less polar nature of a molecule. By way of examples, the following solvents have a Hildebrand total solubility parameter of:
7.3 for hexane (apolar solvent),
11.6 for butylene glycol, 12.7 for ethanol and 16.5 for glycerol (polar solvents),
23.4 for water H₂O (polar solvent).

The preferred polar solvents are those consisting of a compound containing at least one polar covalent bond of the 0-H type. As a particularly preferred polar solvent, one solvent or a mixture of solvents will be used, selected from water, C₁—O₄ alcohols, such as ethanol, glycols, such as ethylene glycol, glycerol, butylene glycol and propylene glycol, and mixtures thereof. Advantageously, water, ethanol or mixtures thereof will be used. Advantageously, water will be used as an extraction solvent (eco-extraction).

In particular, the plant material is extracted using a solvent consisting of water, with or without a pH modifier (e.g. organic acid, citric acid, hydrochloric acid), without any other additional solvent, in a ratio of 10 g plant material per 100 g water (referred to as 10% plant material load). The plant material load can be between 5 and 30%, preferably 10%.

Extraction can be carried out at high temperature by reflux or by maceration at room temperature.

Extraction can be done using well-known physicochemical techniques such as ultrasound, microwave, extrusion, pulsed electric field, and/or cryoextraction.

In the case of an ultrasound extraction, the duration of the extraction may range from 2 to 10 minutes, in particular 5 minutes.

In the case of an extraction using another technology among those mentioned above, the duration may generally range from 1 h to 10 h, in particular 2 to 6 h, or even 4 h, in particular at 60° C.

The extraction process advantageously includes a filtration step to separate the liquid phase from the depleted plant material. The skilled person will choose suitable filtration sieves, in particular with filtration diameters from 0.1 μm to 0.5 μm, in particular from 0.2 to 0.3 μm.

The cycle of extraction and filtration can be repeated several times in order to deplete the plant material of substances with an affinity for the extraction solvent.

The extraction process can be further completed by a step of partial or total removal of the extraction solvents.

The extract can be advantageously concentrated by removing a portion of the solvent or extraction solvent mixture.

It is thus possible to obtain an aqueous concentrate free of a significant amount of organic solvents or, by removing all the extraction solvent, to obtain a dry residue.

Alternatively, the product of the extraction step can be freeze-dried or atomized to form a powder.

According to a particularly preferred embodiment, the aqueous extract of rose fruit according to the invention is obtained by a process that increases the extraction yield and enriches the extract with water-soluble products usually contained in plant juices, in particular sugars, mineral salts, proteins, which are beneficial for the micronutrition of the skin.

Thus, according to a particular and preferred embodiment of the invention, the aqueous extract of rose fruit is obtained by the extraction process comprising the following steps:
a) harvesting fresh rose fruit, optionally stored at −20° C.,
b) grinding the fruit
c) mixing 5 to 30%, in particular 10% of plant material in preferably acidified water (e.g. citric acid) at a temperature ranging notably from 10 to 60° C., in particular 30° C.
d) gradually increasing the temperature of the mixture, in particular from 30 to 100° C., notably up to 50 to 70° C., or even up to 60° C.
e) extraction optionally under stirring,
f) liquid/solid separation,
g) optional addition of preservatives,
h) filtration, in particular to 0.22 μm,
i) optionally packaging, under nitrogen if necessary,
j) optionally storage at +4° C.

The rose fruits are selected from ‘green’ fruit, ‘intermediate’ fruit, ‘ripe’ fruit and mixtures thereof.

According to a particular embodiment, ‘green’ fruit is used.

According to another embodiment, ‘intermediate’ and/or ‘ripe’ fruit are used.

As preservatives in step g), particular mention may be made of sodium benzoate, potassium sorbate, phenoxyethanol and mixtures thereof.

According to a particular and preferred embodiment, the aqueous extract of rose fruit according to the invention is an aqueous extract of fruit of the Evanrat or Jardin de Granville® rose variety obtained according to the extraction process described above, otherwise known as ‘Aqueous Extract of rose fruit’ and comprises 1 to 2% by weight of dry matter (active ingredient) of extract of rose fruit, 97.5% to 98.5% water.

According to a particular and preferred embodiment, the aqueous extract of rose fruit according to the invention, is an aqueous extract of fruit of the Evanrat or Jardin de Granville® rose variety obtained according to the extraction process described above, otherwise called ‘Aqueous extract of rose fruit’ in the illustrative examples described below, and comprises 1 to 2% by weight of dry matter (active ingredient) of extract of rose fruit, 97.5% to 98.5% water, 0.5% citric acid, and qs preservatives.

According to a particular embodiment, the aqueous extract of rose fruit comprises 1 to 2% by weight of dry matter (active ingredient) of extract of rose fruits and rose flowers (in a mass ratio of 0.01 flowers per 9.99 fruit), 97.5% to 98.5% water, 0.5% citric acid, and qs preservatives.

According to a particular and preferred embodiment of the invention, the cosmetic composition according to the invention is characterized in that the aqueous extract of rose fruit comprises an extract of rose fruit in a polar solvent, in particular in a weight ratio of 10:90 (plant extract:polar solvent). The aqueous extract of rose fruit according to the invention, in particular obtained according to the method described above, advantageously comprises micronutrients (calcium, potassium) and macronutrients such as plant sugars (glucose and fructose, and possibly saccharose).

According to a particular and preferred embodiment, the aqueous extract of rose fruit according to the invention, in particular of the Evanrat or Jardin de Granville® rose variety, includes a total mineral content ranging from 300 to 1000 ppm, in particular from 400 to 800 ppm and including in particular calcium and potassium.

The Applicant has shown that the mineral composition (calcium+potassium) of the aqueous extract of rose fruit according to the invention was much higher than that of an aqueous extract of rose flowers ‘Cryoextract’ described below (610 ppm vs. 220 ppm).

According to a particular and preferred embodiment, the aqueous extract of rose fruit according to the invention, in particular of the Evanrat or Jardin de Granville® rose variety, has a total sugar content ranging from 1 to 3%, and notably including fructose and glucose and possibly sucrose.

The Applicant also showed that the plant sugar-rich aqueous extract of rose fruit according to the invention, applied at 0.3% on cultured keratinocytes, made it possible to obtain an increase in ATP synthesis of +21%.

The content of aqueous extract of rose fruit in the final cosmetic composition will generally be from 0.1% to 95%, in particular from 0.5% to 80% by weight of raw material based on the total weight of the composition. According to a first embodiment, it is present in a content ranging from 50% to 95%, in particular from 60% to 90% by weight of raw material based on the total weight of the composition. According to another particular embodiment, it is present in a content ranging from 0.1% to 20%, preferably from 0.5% to 10%, and more preferably from 1% to 5% by weight of raw material based on the total weight of the composition.

For a raw material comprising 1% by weight of dry extract of rose fruit, this is equivalent to 0.001% to 0.02% by weight, in particular 0.005% to 0.01%, and in a particular embodiment 0.01% to 0.05% by weight of dry (active) matter based on the total weight of the composition.

Galenic

The cosmetic composition of the invention may be in any galenic form suitable for topical application to the skin, for example a serum, an oil-in-water emulsion, a water-in-oil emulsion, a multiple emulsion, or an aqueous gel.

The composition is preferentially intended to be applied to the face and is provided for example in the form of a lotion, a care cream, a serum, a facial fluid, a care gel for the face and in particular the eye contour area. According to a particular embodiment, the composition is intended to be applied to the contour of the eyes and is presented in the form of a serum, a cream or an eye contour gel, preferentially an aqueous gel for the eye contour.

According to a particular embodiment, the cosmetic composition according to the invention is in the form of an aqueous gel for the eye contour. This galenic formulation in gel form is particularly adapted to the eye contour, since it confers a fresh effect that is favorable to the decongestion of bags and dark circles, a fresh effect that can be amplified by the contact of the applicator if the composition of the invention is applied to the skin with an applicator as described below.

The aqueous phase generally represents 1 to 99% by weight, based on the total weight of said composition.

Aqueous Phase

The aqueous phase of the composition according to the invention comprises water and optionally a water-soluble solvent.

‘Water-soluble solvent’ according to the invention means a compound which is liquid at room temperature and miscible with water (miscibility in water of more than 50% by weight at 25° C. and atmospheric pressure). Particular mention may be made of:
lower mono-alcohols C₁-C₅ such as ethanol, isopropanol and mixtures thereof;
C₂-C₈ glycols such as ethylene glycol, propylene glycol, 1,3-butylene glycol, dipropylene glycol, and mixtures thereof;
C₂-C₃₂ polyols such as polyglycerols, polyethylene glycols, and mixtures thereof, and mixtures thereof.

It may also include hydrophilic gelling agents, antioxidants, preservatives and mixtures thereof. By way of hydrophilic gelling agents, particular mention may be made of acrylic acid polymers such as those with the INCI name Carbomer or the commercial product Carbopol®, copolymers of acrylic and methacrylic acid, carboxyvinyl polymers, associative thickeners of acrylic or polyurethane type, polysaccharide gelling agents such as alginates, natural or modified gums such as xanthan gums, carrageenan gums, agar gums, guar gums, gellan gums, chitosans, mannans, pullulans, cellulose derivatives, gelatin, pectins, mineral gelling agents such as bentones or modified silicas, and mixtures thereof.

Natural or modified gum means an optionally modified polysaccharide which hydrates in an aqueous medium to form a viscous solution or dispersion. Natural gums include seaweed extracts, plant exudates and gums extracted from plant seeds or roots and those obtained by microbiological fermentation. Modified or semi-synthetic gums include cellulose and starch derivatives and, in general, derivatives of all natural gums.

According to a particular embodiment, the cosmetic composition of the invention comprises an aqueous phase which is gelled, in particular by the presence of at least one acrylic polymer, in particular an acrylic acid polymer or copolymer of acrylic and methacrylic acid.

As examples of acrylic polymers, particular mention may be made of:
the polymer with the INCI name ‘carboxyvinylpolymer’ marketed under the name Carbopol 980;
the polymer with the INCI name ‘hydroxyethyl acrylate/sodium acryloyldimethyl taurate copolymer’ marketed under the name ‘Sepinov™ EMT 10’ by the firm SEPPIC;
the polymer with the INCI name ‘sodium acrylate copolymer and lecithin’ marketed under the name Lecigel™ by the firm Lucas Meyer Cosmetics,
   and mixtures thereof.

According to a particular and preferred embodiment, a copolymer of acrylic and methacrylic acid will be used, in particular the polymer with the INCI name ‘sodium acrylate copolymer and lecithin’ marketed under the name Lecigel™ by the firm Lucas Meyer Cosmetics,

According to another particular embodiment, the polymer with the INCI name ‘hydroxyethyl acrylate/sodium acryloyldimethyl taurate copolymer’ marketed under the name ‘Sepinov™ EMT 10’ by the firm SEPPIC will be used.

The content of hydrophilic gelling agent(s) in the cosmetic composition of the invention will generally be from 0.5 to 5%, in particular from 0.7 to 4%, preferably from 0.9 to 3% and more preferably from 1 to 2% by weight based on the total weight of said composition.

Fat or Oil Phase

The cosmetic composition of the invention may also include a fat (solid fat) or oil phase.

“Oil phase” means an oil or a mixture of oils that are miscible with each other. “Oil” means, in the sense of the invention, a fatty substance, not soluble in water, liquid at 25° C. and 0.1 MPa, and preferably non-volatile, having a vapor pressure, at 25° C. and 0.1 MPa, not zero less than 2.6 Pa, preferably less than 0.13 Pa.

An oil phase according to the invention may comprise hydrocarbon, silicone, fluorinated or non-fluorinated oils, and mixtures thereof.

These oils can be volatile or non-volatile, vegetable, mineral or synthetic.

According to the invention, ‘hydrocarbon oil’ means an oil containing mainly hydrogen and carbon atoms.

According to the invention, ‘silicone oil’ means an oil containing at least one silicon atom, in particular at least one Si—O group.

According to the invention, ‘fluorinated oil’ means an oil containing at least one fluorine atom.

As non-volatile hydrocarbon oils, particular mention may be made of hydrocarbon oils, hydrocarbon oils of vegetable origin, C₁₀-C₄₀ synthetic ethers, C₁₀-C₄₀ synthetic esters, C₁₂-C₂₆ fatty alcohols, C₁₂-C₂₂ higher fatty acids, and mixtures thereof.

As non-volatile silicone oils, particular mention may be made of phenylated silicone oils, non-phenylated silicone oils, and mixtures thereof.

The oils may be present in the composition of the invention in a content ranging from 0.01 to 95% by weight based on the total weight of the composition. According to a particular embodiment, the oils will be present in a content less than or equal to 10%, in particular less than or equal to 5%, or even less than or equal to 1% by weight based on the total weight of the composition. According to a particular embodiment, the cosmetic composition of the invention does not include oils, apart from possible lipophilic compounds provided by the ingredients of the composition such as lecithin (phospholipid) provided by the gelling agent Lecigel™.

The fat or oil phase may also include lipophilic gelling agents, film-forming polymers, surfactants, antioxidants and mixtures thereof.

According to a particular embodiment, the cosmetic composition of the invention is in the form of an aqueous gel comprising an aqueous phase, at least one hydrophilic gelling agent chosen from polymers of acrylic acids, polysaccharides and mixtures thereof.

According to a particular embodiment, the cosmetic composition is in the form of an aqueous gel intended in particular for the contour of the eyes comprising at least:
an aqueous extract of rose fruit according to the invention, in particular an aqueous extract of fruit of the Evanrat or Jardin de Granville® rose variety as described above, in particular in a content ranging from 1 to 10% by weight based on the total weight of the composition;
an aqueous phase representing from 80% to 99% by weight of the composition and comprising at least one hydrophilic gelling agent, chosen in particular from polymers of acrylic acids, polysaccharides, and mixtures thereof, and
an oil phase representing less than 2% by weight of the composition.

According to a particular embodiment, the cosmetic composition of the invention does not include any rose extract other than rose extracts of the Evanrat or Jardin de Granville® rose variety.

According to a particular embodiment, the cosmetic composition of the invention comprises at least one other rose extract of the Evanrat or Jardin de Granville® rose variety, chosen from an aqueous extract of rose flowers, an oil extract of rose flowers, and their mixture, and preferably:
an aqueous extract of rose flowers of the Evanrat or Jardin de Granville® rose variety obtained by the cryoextraction process described in application EP0425391; in particular an extract comprising 0.5% dry matter in 99.5% of a water/glycerol mixture (named ‘Cryoextract’ in the illustrative examples) and
an oil extract of rose flowers of the Evanrat or Jardin de Granville® rose variety obtained by the dynamic enfleurage process as described in application WO2010/112760; in particular an extract comprising 0.5% to 1.5% dry matter in 98.5-99.5% organic deodorized oleic sunflower oil (named ‘Satin oil’ in the following illustrative examples).

The addition of these aqueous and oil extracts of rose flowers, particularly of the Evanrat or Jardin de Granville® rose varieties, provides additional micronutrients, particularly essential fatty acids (omega 3, 6 and 9) provided by the oil extract of rose flowers and minerals (manganese, magnesium, copper, zinc, etc.) provided by the aqueous extract of rose flowers.

The Applicant further showed in a previous study that said aqueous extracts of rose flowers and oil extract of rose flowers stimulate the expression of epidermal clock genes, and the expression of epidermal clock target genes, involved in particular in lipid metabolism, skin barrier, cell differentiation, cell communication and/or cell cohesion.

In particular, the aqueous extract of rose flowers stimulates the expression of the clock genes Cryptochrome Circadian Clock 2 CRY2, Period 1 PER1 and Period 3 PER3 and the oil extract of rose flowers stimulates the expression of the clock gene Period 2 PER2.

The aqueous extract of rose flowers stimulates the expression of the Keratin 10 KRT10 and Desmoglein 1 DSG1 genes and the oil extract of rose flowers stimulates the expression of the Ceramide-synthase 3 CERS3, Calmodulin 3 CALM3, Keratin 1 KRT1, Gap-junction alpha-1 protein GJA1/Connexin 43 C×43 and Desmocollin 3 DSC3 genes.

The use of these extracts of rose flowers thus makes it possible to obtain effects complementary to those related to the aqueous extract of rose fruit according to the invention.

The composition can further include a rose fragrance.

Additional Ingredients

The composition of the invention may also include any additive commonly used in cosmetics such as UV filters, antioxidants, perfumes, cosmetic active agents, such as emollients, moisturizers, vitamins, anti-aging agents, lightening agents, fillers, pearlescent agents and mixtures thereof.

As cosmetic active agents, it may include microcirculation-promoting agents intended to reduce bags under the eyes, such as extracts of Centella asiatica, extract of horse chestnut (Aesculus hippocastanum), etc.

It may also include fillers and/or mother-of-pearl, in particular white mother-of-pearl, intended to mask color imperfections (dark circles) and illuminate the eyes.

Applicator

The cosmetic composition according to the invention is advantageously applied to the eye contour area. The application can be made with the finger or advantageously with an applicator, which will allow a more precise application on the eye contour and/or an improved effectiveness in connection with a better penetration of the cosmetic active ingredients and/or a complementary mechanical action related to the movements of the applicator.

The applicator may include a massaging head consisting of one or more identical balls, with a rotation of 180 to 360°, preferably 360°. The massage surface is increased compared with a finger application, the drainage of the bags is more efficient than with a single-ball roll-on. Associated with a massage technique in a back and forth movement from the temple to the corner of the eye, this applicator reactivates microcirculation and cellular oxygenation and amplifies the assimilation of the micronutrients of the cosmetic composition of the invention.

Cosmetic Process

The invention also relates to a cosmetic process intended to promote and/or improve the supply of nutrients to the skin, promote and/or improve epidermal cohesion and/or differentiation, promote and/or improve the barrier function, densify the extracellular matrix and/or reduce its degradation, improve the elasticity and/or firmness of the skin, in particular the eye contour, prevent and/or reduce dark circles and/or bags around the eyes, improve the radiance and/or homogeneity of the skin, in particular the eye contour area, promote and/or improve hydration, and/or prevent and/or reduce the formation of wrinkles and/or fine lines, comprising the application to the skin, in particular the face and/or neck and preferably the eye contour area, of a cosmetic composition as defined according to the invention comprising at least one aqueous extract of rose fruit according to the invention, in particular of the Evanrat or Jardin de Granville® rose variety.

The composition can be applied to the body, face and/or neck. According to a particular embodiment, the composition is applied to the face and/or neck. According to a particular and preferred embodiment, the composition is applied to the eye contour.

According to a particular embodiment, the invention relates to a cosmetic process intended to densify the extracellular matrix and/or reduce its degradation, comprising the application to the eye contour area of a cosmetic composition as previously defined according to the invention. According to a particular embodiment, the invention relates to a cosmetic process intended to improve the elasticity and/or firmness of the skin, comprising the application to the eye contour area of a cosmetic composition as previously defined according to the invention.

According to a particular and preferred embodiment, the invention relates to a cosmetic process intended to improve the radiance and/or the homogeneity of color of the eye contour, comprising the application to the eye contour of a cosmetic composition as previously defined according to the invention.

According to a particular and preferred embodiment, the invention relates to a cosmetic process intended to prevent and/or diminish dark circles and/or bags around the eyes, comprising the application to the eye contour area of a cosmetic composition as previously defined according to the invention.

Preferably, a cosmetic composition comprising an aqueous extract of rose fruit according to the invention, alone or in combination with other extracts of rose flowers of the Evanrat or Jardin de Granville® rose variety as described above, will be used in these cosmetic processes.

Cosmetic Uses

The invention also relates to the non-therapeutic cosmetic use of at least one effective amount of at least one aqueous extract of rose fruit according to the invention, in particular of the Evanrat or Jardin de Granville® rose variety, as an agent intended to promote and/or improve epidermal cohesion and/or differentiation, and to promote and/or improve the barrier function, densify the extracellular matrix and/or reduce its degradation, improve the elasticity and/or firmness of the skin, particularly around the eyes, prevent and/or reduce dark circles and/or bags around the eyes, improve the radiance and/or homogeneity of the skin, particularly around the eyes, promote and/or improve hydration, and/or prevent and/or reduce the formation of wrinkles and/or fine lines.

The aqueous extract of rose fruit used according to the invention is as described above. Preferably, an aqueous extract of rose fruit of the Evanrat or Jardin de Granville® rose variety obtained by the process of cryogrinding and hot extraction of the fresh fruit of Jardin de Granville® rose will be used; in particular an extract comprising 1% dry matter in 97.5% water, 0.5% citric acid and qs preservatives (called ‘Aqueous extract of rose fruit’ in the illustrative examples).

The invention will now be illustrated in the following non-limiting examples.

### EXAMPLES

Materials and Methods

Rose fruit of the Evanrat variety, in particular fruit of the Jardin de Granville® rose, available from nurseries, will be used as the plant material used to obtain the aqueous rose extracts shown in these examples.

Aqueous Extract of Rose Fruit According to the Invention

Rose fruit of the Evanrat variety, in particular fruits of the Jardin de Granville® rose available from nurseries, are used as plant material.

The aqueous extract of rose fruit is obtained by the cryogrinding and hot extraction process (60° C.) in water described above. The aqueous extract of rose fruit is obtained comprising 2% by weight of dry matter (active ingredient), 97.5% by weight of water, 0.5% citric acid and preservatives qs 100%.

Other rose extracts, in particular of the Evanrat variety, preferably the Jardin de Granville® rose, can be used in association with the aqueous extract of rose fruit according to the invention to provide complementary effects:
Aqueous extract of rose flowers (‘Cryoextract’)

Rose flowers (petals) of the Evanrat variety, in particular Jardin de Granville® rose flowers available at nurseries, are used as plant material.

The aqueous extract of rose flower is obtained using the cryoextraction process described above, in particular according to the process described in patent application EP0425391. A Cryoextract is obtained comprising 0.5% by weight of dry matter (active ingredient), 49-50% by weight of water, 49% by weight of glycerol and preservatives qs 100%. The INCI name of this aqueous extract of rose is Water, Glycerin, Rose Extract or Rosa Hybrid Flower Extract, Water, Glycerin.
Oil extract of rose flowers (‘Satin Oil’)

Rose flowers (petals) of the Evanrat variety, in particular Jardin de Granville® rose flowers available at nurseries, are used as plant material.

The oil extract of rose flower is obtained according to the dynamic enfleurage process described above, in particular according to the process described in the patent application WO2010/112760. An oil extract Satin Oil is obtained comprising 0.5-1.5% by weight of rose dry matter (active ingredient), and 98.5-99.5% by weight of deodorized organic oleic sunflower oil. The INCI name of this oil extract of rose is Rose extract and Helianthus annuus (sunflower) seed oil or Rosa Hybrid Flower Extract, Helianthus annuus (sunflower) seed oil.

The impact of the aqueous extract of rose fruit on cellular energy (ATP synthesis), the expression of genes involved in epidermal cohesion and differentiation (barrier function), the densification of the extracellular matrix, and cellular permeability were studied.

### Example 1: Effect of the Aqueous Extract of Rose Fruit on ATP Synthesis

The effect of the aqueous extract of rose fruit on ATP synthesis in cultured normal human keratinocytes (NHK) was tested.

NHK were treated for 24 hours with:
Aqueous extract of rose fruit: 0.3%.
Positive control: Early Boost© from CODIF as energizer (extract of Jania rubens rich in taurine associated with an oligofurcellaran): 0.15%.

After 2 hours of treatment, the level of ATP production by bioluminescence is detected and measured using the kit from the firm Biovision marketed under the name ApoSENSOR™ ATP Cell Viability Bioluminescence Assay Kit, according to the protocol and the recommendations of the supplier.

This assay uses luciferase to catalyze the formation of light from ATP and luciferin according to the following reaction:

ATP+luciferin+O2→Oxyluciferin+AMP+PPi+Co2+light.

The light produced is measured with a luminometer (Omega, BMG) and is proportional to the amount of ATP synthesized.

A standard curve from 0.001 μg/mL to 1000 μg/mL is defined for the intracellular ATP concentration.

After 2 hours of treatment, it is observed that that the aqueous extract of rose fruit at 0.3% significantly increases the synthesis of ATP (+21%). The positive control gives the expected result (+32%) and validates the experiment.

### Example 2: Effect of the Aqueous Extract of Rose Fruit on the Strengthening of the Barrier Function (Differentiation and Epidermal Cohesion)

Normal human keratinocytes (NHK) are cultured and then treated with the aqueous extract of rose fruit described in the Materials and Methods section.

After treatment of the NHK, a TaqMan Low Density Array (TLDA) study on the genes studied is performed using the cDNAs obtained after reverse transcription of the total RNAs extracted. The aqueous extract of rose is tested at 0.15% and 0.31% by weight of raw material (excipient water).

NHK Culture

Normal human keratinocytes are derived from a skin sample from plastic surgery. The cells are cultured the complete Epilife medium at P5 with a seeding density of 50,000 cells per well, in 12-well plates. At subconfluence, the cells are treated 24 hours with the doses of rose extract described above.

TaqMan Low Density Array (TLDA) Technology
Obtaining total RNAs

The cell culture medium is removed and 250 μL of RLT lysis buffer (provided in the Nucleospin RNA trace kit, Macherey-Nagel) is added. The cells are scraped with a Cell Scraper and then the cell lysate is recovered in a 1.2 mL deepwell (provided in the Nucleospin RNA kit). The total RNAs are extracted according to the defined protocols.

The total RNA solutions obtained are assayed, and their quality checked, using a microplate reader, the spectrostarNANO (BMG Labtech) coupled to the MicrolabSTAR. This apparatus is connected to the computer controlling the Robotics platform and has the specific software for the analysis of the results (MARS software). The technique requires a 384-well microplate (LoBase), a positive control (RNA 250, AM7155, Thermofisher) to validate the pipetting performed by the robot as well as the values generated by the spectrostarNANO reader.
Synthesis of complementary DNA

The reverse transcription (RT) kit used is the High Capacity Reverse Transcription Kit (Thermo Fisher). It was used according to the protocol provided. 500 ng of total RNA is diluted in water for a final volume of 25 μL. It is then incubated for 10 minutes at 25° C. then 2 hours at 37° C. in the presence of 25 μL of High Capacity Reverse Transcription Kit 2× reaction mixture prepared beforehand as indicated below. The different incubations are performed in the TRobot (Biometra).

TABLE 1
High Capacity Reverse Transcription Kit
2X reaction mixture for 1 reaction

| | Reagents | | | | | |
|---|---|---|---|---|---|---|
| | RT buffer | dNTP | Primer | RNase OUT | RT | H2O |
| Volume | 5 μL | 2 μL | 5 μL | 0.5 μL | 2.5 μL | 1 μL |

PCR-TaqMan Low Density Array

15 μL of each RT is mixed with 60 μL of water and then 75 μL of TaqMan Gene Expression master mix (ThermoFisher item number 4369510), containing the DNA polymerase, is added. After homogenization, 100 μL is deposited on the microfluidic cards containing the probes corresponding to the genes tested (Table 2 below), and these are centrifuged and sealed. The CD Rom corresponding to the profile of the genes deposited on the plates is loaded into the SDS 2.3 software, specifying the location of each gene on the card. The control (or “endogenous”) gene to be used for normalization of results is to be indicated before starting the PCR. The latter is performed according to the protocol provided by Applied Biosystems in the ABI Prism 7900HT Sequence detection system. The qPCR steps are 2 min at 50° C., 10 min at 94.5° C. then 30 min at 97° C. and 1 min at 59.7° C. for 40 cycles.

TABLE 2
List of genes on the microfluidic card.

| | | | RefSeq |
|---|---|---|---|
| | | | GeneBank |
| | | TaqMan part | accession |
| Gene | Symbol | number | number |
| Transglutaminase 1 | TGM1 | Hs00165929_m1 | NM_000359 |
| Small Proline-Rich | SPR1B | Hs00824893_m1 | NM_003125 |
| Protein 1B (Cornifin) | | | |
| Cytokeratin 1 | KRT1 | Hs00196158_m1 | NM_006121.3 |
| E-Cadherin | CDH1 | Hs00170423_m1 | NM_001317184.1 |
| Connexin 43 | GJA1 | Hs00748445_s1 | NM_000165.4 |
| Desmoglein | DSG1 | Hs00170047_m1 | NM_001942.3 |
| Occludin | OCLN | Hs00170162_m1 | NM_001205254.2 |
| Claudin-1 | CLDN1 | Hs00221623_m1 | NM_021101.4 |

Statistical Analysis

Real-time quantitative PCR can be exploited if its efficiency is between 90% and 110%. For each sample, the number of cycles at which the signal appears is determined by the SDS 2.3 software. For the same assay, the expression levels of the transcripts of interest obtained are normalized to the value obtained for the household gene Beta-2-microglobulin. This gene, whose expression is constitutive and invariant, makes it possible to avoid any variations induced during the experiment (total RNA assay, pipetting, reverse transcription step, PCR in the apparatus).

In the TLDA RT-PCR method, quantification is performed using the comparative method of ΔΔCt. The relative quantification (RQ) values obtained correspond to the amplitude level (x times more or less than the control) of the expression compared with our control, here the non-irradiated. The RQ is obtained by the following calculation where the control is equal to 1:

RQ=2^{-ΔΔCt}=2^{-(ΔCt treated-ΔCt untreated)}
ΔCt treated=Ct treated target gene−Ct treated household gene
ΔCt untreated=Ct untreated target gene−Ct untreated household gene
In order to evaluate statistically significant variations in transcriptional activity, we will use the Student's t-test. Each condition is performed in triplicate (3 untreated and 3 treated under the same conditions). Fischer's F-test is first applied by comparing the two data matrices. When the value is greater than α=0.05 then the variance for the Student's t-test is 2, when the Fischer's F-test is less than α=0.05 then the variance will be equal to 3. The transcriptional variations selected will be those that have a Student's t-test lower than α=0.05.

Results are presented on average over n=3. Student's t-test was used to compare the effect between treated and untreated cells.

Results are considered significant for p<0.05(*) or p<0.01(**).

The results are presented in Table 3 below:

TABLE 3

| | | | Extract* | Extract* |
|---|---|---|---|---|
| Gene Type | Gene | Symbol | 0.15 | 0.31% |
| Differentiation/Skin | Transglutaminase 1 | TGM1 | 2.148 | 2.716 |
| barrier/Fats/ | | | | |
| Desquamation | | | | |
| | Small Proline-Rich | SPRR1B | 1.782 | 2.272 |
| | Protein 1B | | | |
| | (Cornifin) | | | |
| | Cytokeratin 1 | KRT1 | 2.207 | 2.641 |
| Adhesion/ | E-Cadherin | CDH1 | — | 1.516 |
| Cohesion/ | | | | |
| Communication | | | | |
| | Connexin 43 | GJA1 | 1.489 | 2.199 |
| | Desmoglein | DSG1 | 1.832 | 3.139 |
| | Occludin | OCLN | 1.430 | 2.319 |
| | Claudin-1 | CLDN1 | — | 2.072 |

*aqueous extract of rose fruit described in the Materials and Methods section

At the dose of 0.31%, for each marker, this corresponds to a respective increase of:

TABLE 4

| | |
|---|---|
| Transglutaminase | +172% |
| SPRR 1B * | +127% |
| Small Proline-Rich | |
| Protein 1B | |
| Keratin 1 | +164% |
| Cadherin 1 | +52% |
| Connexin 43 | +120% |
| Desmoglein 1 | +214% |
| Occludin | +132% |
| Claudin 1 | +107% |

These results show that the aqueous extract of rose fruit according to the invention stimulates the expression of genes involved in epidermal cohesion and differentiation.

And the effect of the aqueous extract of rose fruit (0.31% by weight of raw material equivalent to 52.7 μg/mL(/DM)) stimulates the expression of the Desmoglein 1 gene (DSG1) with an increase +214% much higher than that obtained in another experiment for a cryoextract of flowers at a slightly higher dose (1% by weight of raw material equivalent to 65 μg/mL(/DM)): +58%; knowing moreover that this cryoextract did not stimulate the other genes.

### Example 3: Effect of the Aqueous Extract of Rose Fruit on the Densification of the Extracellular Matrix

Normal human fibroblasts (NHF) were treated or not with the aqueous extract of rose fruit according to the invention for 24 hours, at a dose of 0.15% (equivalent to 25 μg/ML of dry matter). After treatment of the NHF, a TaqMan Low Density Array (TLDA) study on different genes was performed using the cDNA obtained after reverse transcription of the total RNA extracted from the NHF.

NHF Culture

The fibroblasts are derived from an abdominoplasty performed on a 37-year-old female donor. The cells are inoculated in DMEM 1 g/L glucose medium supplemented with 10% fetal calf serum at P5 with a seeding density of 250,000 cells per well, in 6-well plates. On the day before treatment, the cells are cultured in DMEM 1 g/L glucose medium without fetal calf serum.

NHF Treatment

At confluence, the cells are treated with the aqueous extract of rose fruit, prepared just before use at the final concentration of use in DMEM medium 1 g/L glucose without fetal calf serum. After 24 hours of treatment, the cells are recovered in order to extract the total RNAs.

TLDA (TaqMan Low Density Array)

The TLDA technology study on a gene list is performed using cDNA obtained after reverse transcription of the total RNAs extracted from the NHF according to the protocol described in Example 1.

TABLE 5
List of genes on the microfluidic card

| | | | RefSeq |
|---|---|---|---|
| | | | GeneBank |
| | | TaqMan part | accession |
| Gene | Symbol | number | number |
| Collagen 1 | COL1A1 | Hs00164004_m1 | NM_000088.3 |
| Collagen 3 | COL3A1 | Hs00164103_m1 | NM_000090.3 |
| Collagen 5 | COL5A1 | Hs00609088_m1 | NM_000093.4 |
| Elastin | ELN | Hs00355783_m1 | NM_000201.3 |
| Emilin 1 | Emilin1 | Hs00170878_m1 | NM_007046.3 |
| Fibrillin 1 | FBN1 | Hs00171191_m1 | NM_000138.4 |
| Lysyl oxidase like 1 | LOXL1 | Hs00173746_m1 | NM_005576.3 |
| Procollagen C | PCOLCE | Hs00170179_m1 | NM_002893.3 |
| endopeptidase enhancer | | | |
| Matrix Metalloprotease 3 | MMP3 | Hs00233962_m1 | NM_002422.4 |

The results are presented in Table 6 below:

TABLE 6

| | | | | Vit C |
|---|---|---|---|---|
| | | | | 50 μg/mL |
| | | | Extract* | (positive |
| Gene Type | Gene | Symbol | 0.15% | control) |
| Synthesis- | Collagen 1 | COL1A1 | 16.015 | 3.471 |
| induction | Collagen 3 | COL3A1 | 2.818 | 2.599 |
| | Collagen 5 | COL5A1 | 12.987 | 1.911 |
| | Elastin | ELN | 15.762 | 1.016 |
| Assembly | Emilin 1 | Emilin1 | 5.330 | 2.509 |
| | Fibrillin 1 | FBN1 | 1.947 | 0.989 |
| | Lysyl oxidase like 1 | LOXL1 | 2.995 | 1.202 |
| | LOWL1 | | | |
| | Procollagen C | PCOLCE | 1.450 | 1.156 |
| | endopeptidase enhancer | | | |
| Matrix | Matrix | MMP3 | 0.679 | 1.205 |
| degradation | Metalloproteinase 3 | | | |

*aqueous extract of rose fruit described in the Materials and Methods section

At the dose of 0.15%, for each marker, this corresponds to an increase of:

TABLE 7

| | Extract* | |
|---|---|---|
| | 0.15% | Vit C |
| | (25 μg/mL) | (50 μg/mL) |
| Collagen 1 | +1500% | +247% |
| Collagen 3 | +182% | +160% |
| Collagen 5 | +1200% | +91% |
| Elastin | +1476% | NS |
| Emilin 1 | +433% | +151% |
| Fibrillin 1 | +95% | NS |
| Lysyl oxidase like 1 LOWL1 | +200% | NS |
| Procollagen C endopeptidase enhancer | +45% | NS |
| Matrix Metalloproteinase 3 | −32% | NS |

*aqueous extract of rose fruit described in the Materials and Methods section

These results show that the aqueous extract of rose fruit according to the invention stimulates the gene expression of proteins of the extracellular matrix: Collagen I, III, V, elastin as well as enzymes involved in the formation of fibers: LOX (Lysyl oxidase), PECOL (Procollagen C-endopeptidase enhancer), and that in parallel it inhibits enzymes of degradation of the extracellular matrix (MM P3).

And this effect of the aqueous extract of rose fruit (0.15% by weight of raw material equivalent to 25.5 μg/mL(/DM)) is much greater than that obtained in the same experiment for a cryoextract of flowers at a dose more than 2 times higher (1% by weight of raw material equivalent to 65 μg/mL(/DM)) as shown in the following results:

TABLE 8

| | | | Vit C |
|---|---|---|---|
| | | | 50 μg/mL |
| | Extract* | Cryoextract** | (positive |
| Gene | 0.15% | 1% | control) |
| Collagen 1 | 16.015 | 7.998 | 3.471 |
| Collagen 3 | 2.818 | 1.928 | 2.599 |
| Collagen 5 | 12.987 | 2.960 | 1.911 |
| Elastin | 15.762 | 7.353 | 1.016 |
| Emilin 1 | 5.330 | 1.198 | 2.509 |
| Fibrillin 1 | 1.947 | 0.960 | 0.989 |
| Lysyl oxidase like 1 | 2.995 | 1.177 | 1.202 |
| LOWL1 | | | |
| Procollagen C | 1.450 | 0.888 | 1.156 |
| endopeptidase enhancer | | | |
| Matrix Metalloproteinase 3 | 0.679 | 0.471 | 1.205 |

*aqueous extract of rose fruit described in the Materials and Methods section
**extract rose flower described in the Materials and Methods section

### Example 4: Effect of the Aqueous Extract of Rose Fruit on Vascular Permeability (Anti-Dark Circles/Microcirculation)

In the TLDA study described in Example 1, it was also shown that extract of rose fruit at the dose of 0.15% inhibited by −74% the expression of VEGF, a vascular endothelial growth factor involved in vascular permeability. This effect is particularly advantageous for a use of the extract according to the invention in the eye contour area.

All these results show that the aqueous extract of rose fruit according to the invention, in particular the aqueous extract of rose fruit comprising 1% by weight of dry matter (active ingredient), 97.5% by weight water, 0.5% citric acid and preservatives qs 100%, from the dose of 0.15%:
increases the synthesis of ATP, which promotes the supply of energy to the skin cells;
stimulates gene expression of markers of epidermal cohesion and differentiation that help strengthen the barrier function;
stimulates gene expression of extracellular matrix proteins and inhibits degradation enzymes, participating in the densification of the extracellular matrix, and
inhibits the gene expression of the vascular endothelial growth factor VEGF, participating in a decrease in vascular permeability.

And that these stimulating effects on the barrier function and the extracellular matrix are greater than those obtained with a cryoextract of flowers.

The interest of using an extract of rose fruit according to the invention is therefore understood to promote and/or improve epidermal cohesion and differentiation, strengthening of the barrier function, densification of the extracellular matrix, reduction of cellular permeability, in the skin of the face and particularly in the eye contour area, which is particularly fine and fragile and prone to the formation of dark circles and/or bags.

The extract of rose fruit according to the invention was used, at different concentrations, alone or in combination with other rose extracts with complementary properties, in different cosmetic formulations. The percentages % are expressed in weight of raw material unless otherwise specified.

### Example 5: Cosmetic Formulations

5.1 Composition in the Form of an Emulsion

Aqueous phase:

| | |
|---|---|
| Demineralized water | qs 100.0% |
| Glycols | 20.0% |
| Preservatives | 0.6% |
| Chelator | 0.04% |
| Carbomer (Carbopol ® 981) | 0.3% |
| Sodium polyacrylate (Covacryl ® MV60) | 0.2% |
| Sodium Hydroxide | 0.15% |
| Aqueous extract of rose fruit according to the | 3.0% |
| invention*. | |

Fat phase:

| | |
|---|---|
| Vegetable oil, esters, silicones | 16% |
| Antioxidant | 0.2% |
| Fragrance concentrate | 0.4% |
| Steareth-2 | 0.8% |
| Steareth-21 | 1.5% |

*as described in the Materials and Methods section

The composition is prepared according to the following procedure:
the gelling agents are dispersed in the aqueous phase (excluding aqueous extract of rose fruit and sodium hydroxide) which is heated to 70° C.;
the fat phase (excluding fragrance concentrate, antioxidant) is heated to 70° C.;
the emulsion is made by introducing the fat phase into the aqueous phase under strong stirring;
the gelling agents are neutralized by adding sodium hydroxide and the emulsion is cooled under moderate stirring with the introduction of the fragrance concentrate, the antioxidant and the aqueous extract of rose fruit at low temperature.

Applied to the skin, especially the face, this composition brings nutrition, firmness and hydration to the skin.

5.2 Composition in the Form of a Solid Dispersion of Fatty Substances, of Spherical or Spheroidal Shape

Aqueous phase

| Demineralized water | qs 100% |
|---|---|
| Cryoextract of rose* | 3.00% |
| Aqueous extract of rose fruit | 3.00% |
| according to the invention* | |
| Phenoxyethanol | 0.42% |
| Xanthan gum | 0.36% |

Fat phase

| | |
|---|---|
| C₁₀₋₁₈ triglycerides | 38.84% |
| Satin Oil* | 1% |
| Antioxidant | 0.1% |

*as described in the Materials and Methods section above.

The composition is prepared according to the following procedure:
the wax (C10-18 triglycerides=Lipocire from Gatefossé) is heated above its melting point (a few degrees) with the antioxidant and the oil extract of rose Satin oil;
the melted fat phase is poured under stirring into water previously heated to the same temperature as the fat phase;
the whole is maintained under stirring by a rotary system for a few minutes until the desired droplet size is obtained;
the dispersion obtained is rapidly cooled by adding pre-cooled glycol water (about −4° C.) to solidify the lipid spheroids;
stirring is stopped when the spheroids are solidified and then recovered from the surface or filtered;
the aqueous phase is prepared by mixing water, xanthan gum, preservative and cryoextract of rose, and the aqueous extract of rose fruit according to the invention;
the spheroids are recovered from the surface and incorporated into the xanthan gel containing the rose cryoextract and the aqueous extract of rose fruit according to the invention.

Applied to the skin, this composition confers a nourished, smoother and firmer appearance to the skin.

5.3: Composition in the Form of an Aqueous Gel for the Face

| | |
|---|---|
| Aqueous extract of rose fruit | 95.00% |
| according to the invention * | |
| Preservatives | 0.50% |
| Sugar | 0.20% |
| Carbomer | 0.70% |
| Purified water | Qs 100% |
| EDTA tetrasodium powder | 0.20% |
| Sodium hydroxide | 0.17% |

* as described in the Materials and Methods section

The extract of rose fruit according to the invention and the preservatives are mixed and homogenized at room temperature under stirring. The sugars are added under stirring, then the carbomer, then the water and the EDTA under stirring. The aqueous gel is then neutralized with the addition of sodium hydroxide under stirring until a homogeneous gel is obtained.

Applied to the facial skin, this aqueous gel gives a fresh effect and brings suppleness and firmness to the skin.

5.4: Composition in the Form of an Aqueous Gel

| | |
|---|---|
| Aqueous extract of rose fruit according to the | 80.00% |
| invention* | |
| Phenoxyethanol | 0.50% |
| Glycols | 12.0% |
| Polyol | 2.0% |
| Thickening polymer (INCI Name Hydroxyethyl | 1.60% |
| Acrylate/Sodium Acryloyldimethyl Taurate Copolymer, | |
| Sepinov ™ EMT 10 | |
| BHT | 0.20% |
| Alcohol at 96.2% vol kg | Qs 100% |
| Rose fragrance | 0.30% |

*as described in the Materials and Methods section

The aqueous extract of rose fruit according to the invention, phenoxyethanol, and glycols are mixed under stirring at room temperature; the polyol is then added under stirring. The surfactant Sepinov™ EMT 10 is then added and emulsified in the aqueous phase under stirring, and the alcohol, BHT and rose fragrance are then added.

Applied to the facial skin, this aqueous gel gives a fresh effect and brings suppleness and firmness to the skin.

5.5: Composition in the Form of a Micronutritive Gel-Serum for the Eye Contour Area

| Purified water | Qs 100.00% |
|---|---|
| Glycols | 13.0% |
| Preservatives | 0.60% |
| Carbomer | 0.80% |
| Glyceryl stearate citrate | 0.70% |
| Lecithin and sodium acrylate | 1.20% |
| copolymer (Lecigel PCR negative) | |
| Isostearyl isostearate | 9.0% |
| Bis-diglyceryl polyacyladipate-2 | 1.0% |
| (Softisan 649 MB) | |
| Silica | 2.0% |
| Mother-of-pearl | 1.0% |
| Cryoextract of rose * | 3.0% |
| Extract of Centella asiatica | 0.5% |
| Extract of horse chestnut | 0.1% |
| Aqueous extract of rose fruit | 3.0% |
| according to the invention * | |
| Tocopheryl acetate | 0.10% |
| Rose fragrance | 0.20% |
| Satin oil * | 1.0% |

* as described in the Materials and Methods section

The ingredients of the aqueous phase (water, glycols and carbomer) are mixed at 80° C. under stirring. Then the preservatives are added, then the gelling agents are added at 80° C. with stirring and the temperature is lowered to 75° C. Then the surfactants are emulsified in the aqueous phase under stirring. The fillers and mother-of-pearl, pasted and homogenized beforehand, are added at 40° C. The cryoextract, the aqueous extract of rose according to the invention and the other extracts are then added at 40° C., under stirring to 30° C. After applying this gel-serum to the eye contour area, puffiness and dark circles seem to fade away for a visibly refreshed, illuminated, radiant look. The skin appears nourished, firmer, smoother (reduction in the appearance of fine lines and wrinkles), moisturized and more evenly colored.

1. A cosmetic composition for topical application to the skin comprising, in a physiologically acceptable medium, at least one effective amount of at least one aqueous extract of rose fruit distinct from the fruit of the dog rose (i.e. Rosa canina), or an aqueous extract of rose fruit of the Evanrat or Jardin de Granville® rose variety.

2. The cosmetic composition as claimed in claim 1, wherein the aqueous extract of rose fruit is present in the composition in a content ranging from 0.1% to 95%, or from 0.1 to 20%, or from 0.5% to 10% or from 1% to 5% by weight of raw material based on the total weight of said composition.

3. The cosmetic composition of claim 1, wherein the cosmetic composition is in the form of a solution, an emulsion, a serum or a gel, preferably an aqueous gel for the eye contour area.

4. A cosmetic process intended to promote and/or improve the supply of nutrients to the skin, promote and/or improve epidermal cohesion and/or differentiation, promote and/or improve the barrier function, densify the extracellular matrix and/or reduce its degradation, improve the elasticity and/or firmness of the skin or of the eye contour area, prevent and/or reduce dark circles and/or bags around the eyes, improve the radiance and/or homogeneity of the skin or of the eye contour area, promote and/or improve hydration, and/or prevent and/or reduce the formation of wrinkles and/or fine lines, comprising the application to the skin, in particular the face and/or neck and/or the eye contour area, of a cosmetic composition as defined in claim 1.

5. A cosmetic process for preventing and/or reducing dark circles and/or bags around the eyes and/or improving the radiance and/or homogeneity of the skin around the eyes, comprising the application to the eye contour area of a cosmetic composition as defined in claim 1.

6. Non-therapeutic cosmetic use of at least one effective amount of at least one aqueous extract of rose fruit distinct from the fruit of the dog rose, or an aqueous extract of rose fruit of the Evanrat or Jardin de Granville® rose variety, as an agent intended to promote and/or improve the supply of nutrients to the skin, promote and/or improve epidermal cohesion and/or differentiation, promote and/or improve the barrier function, densify the extracellular matrix and/or reduce its degradation, improve the elasticity and/or firmness of the skin or around the eyes, prevent and/or reduce dark circles and/or bags around the eyes, improve the radiance and/or homogeneity of the skin or around the eyes, promote and/or improve hydration, and/or prevent and/or reduce the formation of wrinkles and/or fine lines.

7. A cosmetic set comprising:
A cosmetic composition as defined in claim 1, and
An applicator suitable for topical application to the skin of the face, especially around the eyes.

8. An aqueous extract of rose fruit of the Evanrat or Jardin de Granville® rose variety obtained using a cosmetically acceptable polar solvent.

9. The aqueous extract of rose fruit of the Evanrat or Jardin de Granville® rose variety as claimed in claim 8, wherein it comprises 1 to 2% by weight rose fruit (active ingredient), 97.5% to 98.5% water, or 1% rose fruit (active ingredient), 97.5% water, 0.5% citric acid and preservatives qs 100%.

10. The aqueous extract of rose fruit of the Evanrat or Jardin de Granville® rose variety as claimed in claim 8, comprising a total sugar content ranging from 1 to 3%, and notably comprising fructose, glucose and possibly sucrose.

11. The aqueous extract of rose fruit of the Evanrat or Jardin de Granville® rose variety as claimed in claim 8, obtained according to the extraction process comprising the following steps:
a) harvesting fresh rose fruit, optionally stored at −20° C.,
b) grinding the fruit
c) mixing 5 to 30%, or 10% of plant material in preferably acidified water at a temperature ranging notably from 10 to 60° C., or 30° C.,
d) gradual increasing the temperature of the mixture, in particular from 30 to 100° C., notably up to 50 to 70° C., or even up to 60° C.,
e) extraction optionally under stirring,
f) liquid/solid separation,
g) optional addition of preservatives,
h) filtration, especially to 0.22 μm,
i) optionally packaging, under nitrogen if necessary,
j) optionally storage at +4° C.
